(19) United States
(12) Patent Application Publication
(10) Pub. No.: US 2014/0077967 A1
(43) Pub. Date: Mar. 20, 2014

(54) WIRELESS PATIENT MONITORING SYSTEM

(71) Applicants: JAVIER ALVAREZ OSUNA, VIGO (ES); JUAN MIGUEL MOURE ALONSO, VIGO (ES); FRANCISCO MARTINEZ RILO, VIGO (ES); ANTONIO ARIAS LOSADA, PONTEVEDRA (ES); SANTIAGO PAN CARNEIRO, VIGO (ES); FRANCISCO ALBERTO ROCHA RIVERA, VIGO (ES); JACOBO CAMPOS CASAL, VIGO (ES); JUAN PABLO BAR RIVEIRO, VIGO (ES); ANDRES IÑIGUEZ ROMO, VIGO (ES); MANUEL VAZQUEZ LIMA, VIGO (ES); CONCEPCION ABELLAS ALVAREZ, VIGO (ES)

(72) Inventors: JAVIER ALVAREZ OSUNA, VIGO (ES); JUAN MIGUEL MOURE ALONSO, VIGO (ES); FRANCISCO MARTINEZ RILO, VIGO (ES); ANTONIO ARIAS LOSADA, PONTEVEDRA (ES); SANTIAGO PAN CARNEIRO, VIGO (ES); FRANCISCO ALBERTO ROCHA RIVERA, VIGO (ES); JACOBO CAMPOS CASAL, VIGO (ES); JUAN PABLO BAR RIVEIRO, VIGO (ES); ANDRES IÑIGUEZ ROMO, VIGO (ES); MANUEL VAZQUEZ LIMA, VIGO (ES); CONCEPCION ABELLAS ALVAREZ, VIGO (ES)

(73) Assignee: IMAXDI REAL INNOVATION SL, VIGO (ES)

(21) Appl. No.: 14/088,364

(22) Filed: Nov. 23, 2013

Related U.S. Application Data
(63) Continuation-in-part of application No. 13/556,076, filed on Jul. 23, 2012.
(60) Provisional application No. 61/515,908, filed on Aug. 6, 2011.

(51) Int. Cl. A61B 5/00; G08C 17/02

(52) U.S. Cl. CPC A61B 5/0026; G08C 17/02
USPC 340/870.01

(57) ABSTRACT

Disclosed embodiments include a wireless portable medical monitoring apparatus that includes (a) a hospital bed or medical stretcher; (b) a plurality of wireless biomedical sensors attached to the hospital bed or medical stretcher; and (c) a communications module configured for wirelessly transmitting jointly compressed biomedical signals. The communication module is configured to transmit signals as a block of coherent data. Additionally, the communication module includes fast-joint coding and decoding, transmission error correction, and information exchange between different layers to optimize network throughput.

### CROSS-REFERENCE TO RELATED APPLICATIONS

This application is a continuation-in-part of U.S. Non-provisional application Ser. No. 13/556,076 filed on Jul. 23, 2012 which claims the priority benefit of U.S. Provisional Application No. 61/515,908 filed on Aug. 6, 2011, which are all incorporated herein by reference in their entirety.

### TECHNICAL FIELD

Disclosed embodiments relate to systems for emergency response. Specifically, they relate to methods, apparatuses, and systems for mobile emergency response.

### BACKGROUND

Recent technological advances enable clinical practitioners to conduct faster diagnosis and treat acute events outside the hospital in emergency response settings. Such diagnosis and treatment requires specialized clinical and communications equipment.

Taking advantage of advances of mobile health technologies (mHealth), biomedical signs can be sent from the emergency vehicles to the hospitals and to mobile devices of specialists in order to accelerate diagnosis, as well as make early preparation for clinical interventions before the patient arrives to the treatment center.

### SUMMARY

Disclosed embodiments include a wireless medical monitoring apparatus that comprises: (a) a hospital bed or medical stretcher; (b) a plurality of wireless biomedical sensors attached to the hospital bed or medical stretcher; and (c) a communications module configured for wirelessly transmitting jointly compressed biomedical signals. According to particular embodiments, and without limitation, the communication module is configured to transmit signals as a block of coherent data. Additionally, in a particular embodiment, the communication module includes fast-joint coding and decoding of said signals, transmission error correction, it is configured to enable information exchange between different layers to optimize network throughput, and adapts the Quality of Service (QoS) guarantees for each type of traffic offered. Each layer in the communications module obtains information features about the channel conditions during transmission and the layer processes are adapted to the conditions during transmission.

### BRIEF DESCRIPTION OF THE DRAWINGS

Disclosed embodiments are illustrated by way of example, and not by way of limitation, in the figures of the accompanying drawings:

FIG. 1 shows a general illustration of the mobile emergency response system according to one embodiment.

FIG. 2 shows a block diagram illustrating the architecture of the mobile emergency response system according to one embodiment.

FIG. 3 shows a block diagram of the patient monitor architecture according to one embodiment.

FIG. 4 shows a block diagram of the cloud infrastructure architecture according to one embodiment.

FIG. 5 shows a block diagram of the cloud medical client architecture according to one embodiment.

FIG. 6 shows a block diagram illustrating the cross-layer interaction according to one embodiment.

FIG. 7 shows a block diagram illustrating the architecture of the progressive source encoder and the channel decoder including rate control according to one embodiment.

FIG. 8 shows a block diagram illustrating the architecture of the cross-layer design (CLD), the context dynamic information (CDI), and the cluster progressive encoder according to one embodiment.

FIG. 9 shows a block diagram to illustrate the security architecture according to one embodiment.

FIG. 10 shows a block diagram to illustrate the architecture of the chaotic video encryption scheme (CVES) according to one embodiment.

FIG. 11 shows a block diagram to illustrate the architecture of the communication module according to one embodiment.

FIG. 12 shows a block diagram to illustrate the UPnP client-server architecture according to one embodiment.

FIG. 13 shows a block diagram to illustrate the pairing method according to one embodiment.

FIG. 14 shows an illustration of the overall system according to one embodiment.

FIG. 15-16 show illustrative block diagrams for interface configuration according to one embodiment.

FIG. 17 shows an illustrative embodiment of the wireless patient monitoring system embedded in a hospital bed or stretcher.

FIG. 18 shows an illustrative embodiment of the wireless patient monitoring system including the Wireless Patient Care Terminal (WPCT) module, the Central Monitoring Medical Unit (CMMU), and the remote Wireless Patient Care Terminal (rWPCT).

FIG. 19 shows a block diagram of the overall architecture according to one embodiment.

FIG. 20A-20B show a detailed block diagram of the system according to one embodiment.

FIG. 21-28 show illustrative aspects of the graphical user interface (GUI) according to particular embodiments

FIG. 29 shows an illustrative GUI in a multi-touch tablet.

### DETAILED DESCRIPTION

The detailed description is divided in two main parts. Part A describes a wireless mobile distributed emergency response monitoring system and the communication methods, architectures and apparatuses that make the system possible. Part B describes a wireless monitoring system which relies on the same methods but is adapted and configured for medical stretchers and hospital beds.

### Part A—Wireless Mobile Distributed Emergency Response Monitoring System & Communication Methods

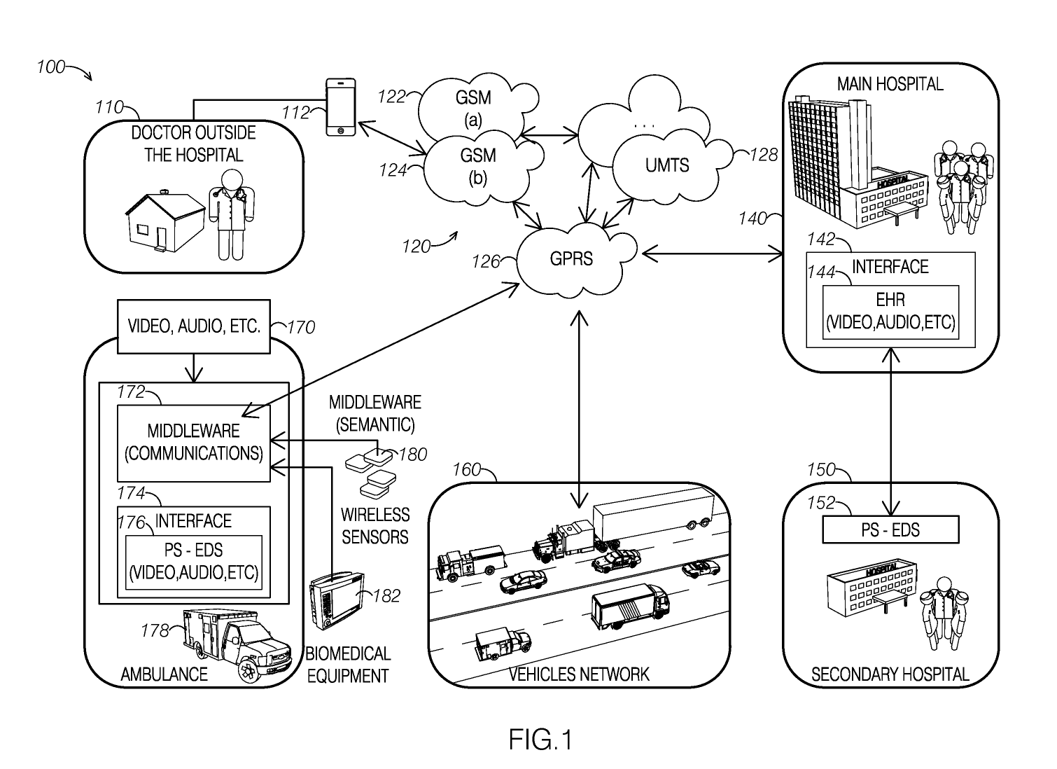

As shown in FIG. 1, disclosed embodiments include a system for mobile emergency response 100 comprising: (a) a patient monitor 302 including 1) an early monitoring apparatus, 2) a multitouch hardware, and a 3) a connectivity platform; (b) a cloud infrastructure for data distribution 402; and (c) a mobile medical client 502.

According to one embodiment, and without limitation, the mobile emergency response system 100 incorporates a monitoring apparatus 302 that includes (a) a plurality of wireless biomedical sensors 180; (b) a connectivity platform 120; (c) a semantic middleware architecture 172; (d) a plurality of biomedical signal processing algorithms; and (e) a security system.

According to one embodiment, the plurality of wireless biomedical sensors include a combination of ECG, NIBP, and SpO2 wireless synchronized sensors 180. These wireless synchronized sensors enable multidata collection and transmission of synchronized and jointly compressed signals. Additionally, the connectivity platform incorporates seamless roaming and includes 1) a location awareness method for vertical mobility management, 2) a handoff method, and 3) a vertical mobility and handoff method especially adapted for packet-switched all-IP. Finally, the emergency response system includes a semantic middleware architecture with an autonomous middleware for ubiquitous and heterogeneous environments. The autonomous middleware for ubiquitous and heterogeneous environments provides semantic interoperability between biomedical devices, security, mobility, context awareness, and quality of service.

Certain specific details are set forth in the above description and figures to provide an understanding of various embodiments disclosed for those of skill in the art. Certain well-known details often associated with computing technology are not set forth in the following disclosure to avoid unnecessarily obscuring the various disclosed embodiments. Further, those of ordinary skill in the relevant art will understand that they can practice other embodiments without one or more of the details described in the present disclosure. Aspects of the disclosed embodiments may be implemented in the general context of computer-executable instructions, such as program modules, being executed by a computer, computer server, or device containing a processor. Generally, program modules or protocols include routines, programs, objects, components, data structures, hardware executable instructions that perform particular tasks or implement particular abstract data types. Aspects of the disclosed embodiments may also be practiced in distributed computing environments where tasks are performed by remote processing devices (processors, microprocessors, computing systems, FPGAs, programable ICs, etc) that are linked through a communications network. In a distributed computing environment, program modules and hardware executable instructions may be located in both local and remote storage media such as memory storage devices (including non-transitory storage media). Those skilled in the art will appreciate that, given the description of the modules comprising the disclosed embodiments provided in this specification, it is a routine matter to provide working systems which will work on a variety of known and commonly available technologies capable of incorporating the features described herein. Additionally, the methods described herein can be implemented in a hardware-readable storage medium (including non-transitory computer-readable media) with an executable program stored thereon, wherein said executable program instructs the processing hardware perform the method steps.

A. General Apparatus and System Overview

According to one embodiment, the system can be used in the same manner as a traditional patient monitor 182. However, the system includes additional hardware with functionality for extending the presentation of the data collected to the remote medical clients. When an accident takes place, the emergency protocol typically calls for placement of biomedical sensors to monitor the patient and control the vital signs. In challenging rescue scenarios where traditional wired monitors 182 are problematic due to the wire limitations, the biomedical wireless sensors 180 can be used.

The data from the wireless sensors 180, as the information coming from the other biomedical equipment installed on the ambulance 178, is connected to a middleware system (with semantic interoperability capabilities) 172 and then transmitted to the hospital 140 and the mobile clients 112 of specialists outside the hospital 110. The proposed mobile emergency system improves communication technologies to perform early monitoring of emergency patients and realize a remote real-time control during the patient transfer through an interface 1174, 142 to the hospital 140 and audio/video communications 170.

Biomedical data transmission takes advantage of existing wireless networks 120 (GSM 122, 124, GPRS 126, UMTS 128, Wifi, WIMAX) with the best signal available at each moment during the emergency vehicle route. This requires a sophisticated vertical handoff method between mobile networks according to a “best connected everywhere” philosophy, that is, it chooses the optimum access network with the Quality of Service (QoS) for the data to be transmitted. In case that connection establishment is not possible based on the above-named networks, the use of vehicular networks 160 is considered. Vehicular networks 160 provide communications among nearby vehicles and between vehicles and nearby fixed equipment.

The ambulance crew that is transferring an emergency patient is remotely connected to the expert team 204 at the hospitals 140, 150 (by video, voice, and with the possibility of consulting the patient PS-EDS) 152, 176, and thus they can follow real-time instructions from experts to stabilize the patient.

According to one embodiment, the hospital staff 204 can participate in a multipoint session with the ambulance 178 crew (within the multi-collaborative environment of the system) receiving the patient's information. The data acquired during emergency transport can be compared in real time with patient's historical clinical data and eventually incorporated in the patient EHR 144 for future use. This multipoint session may be performed by medical specialists from their mobile devices 112 in real time.

According to a particular embodiment, and without limitation, the system is comprised in three main parts: Patient Monitor 302, Medical Cloud 402, and Medical Client 502.
1. Patient Monitor 302: responsible of acquiring, processing, presenting and transmitting the biomedical data. The patient monitoring apparatus comprises the following main structural parts.
   Early monitoring apparatus (biomedical wireless sensors, middleware for semantic interoperability, algorithms for biomedical signal processing and security system).
   A multitouch hardware with a specific embedded system application.
   Connectivity platform (seamless roaming system)
2. Cloud 402: infrastructure for the data distribution
3. Medical Client 502: remote biomedical data viewer on mobile devices.

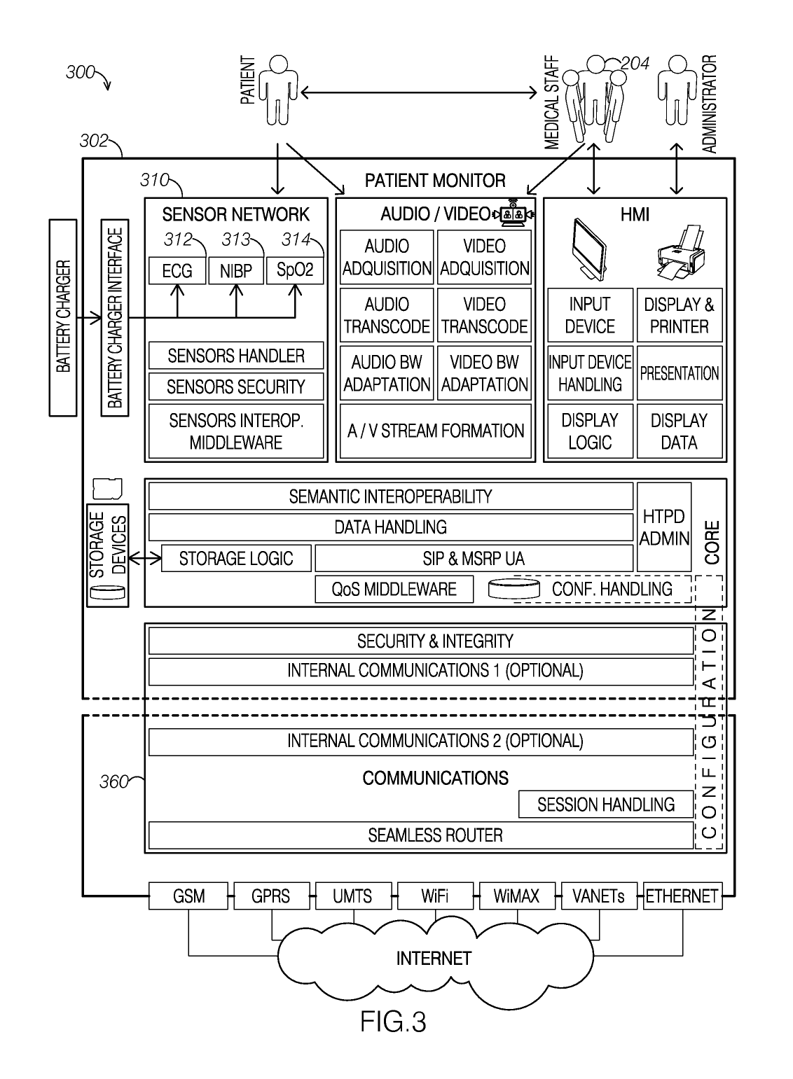

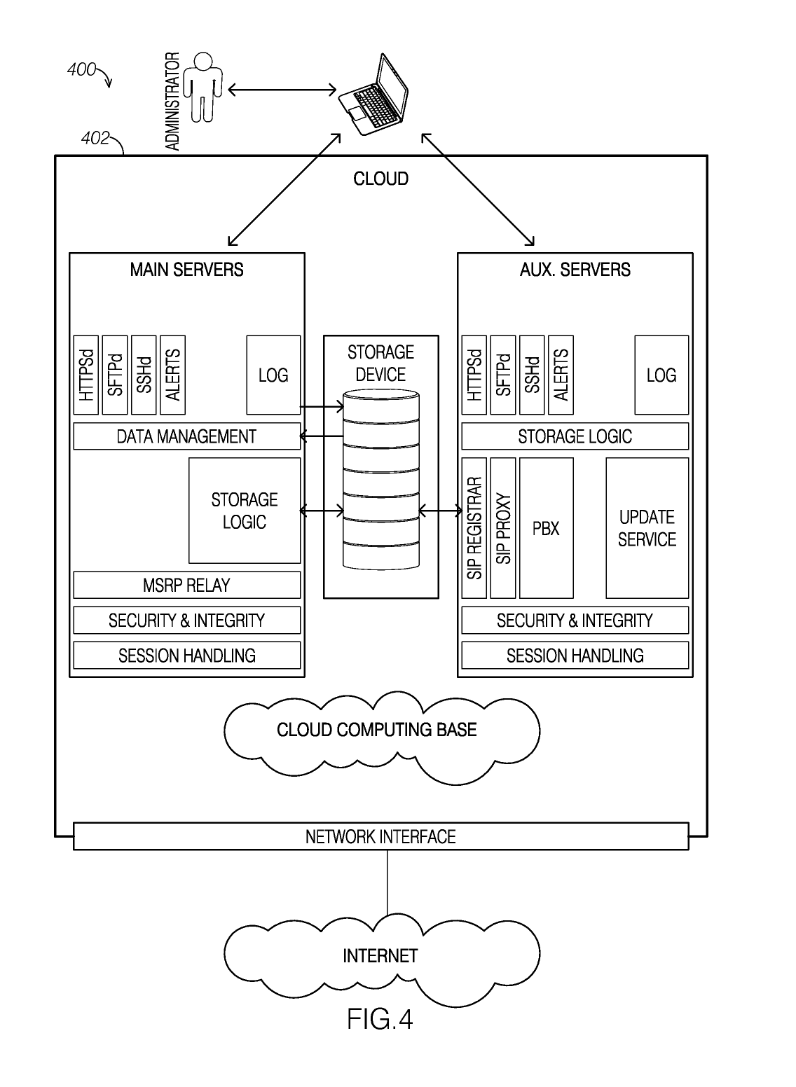

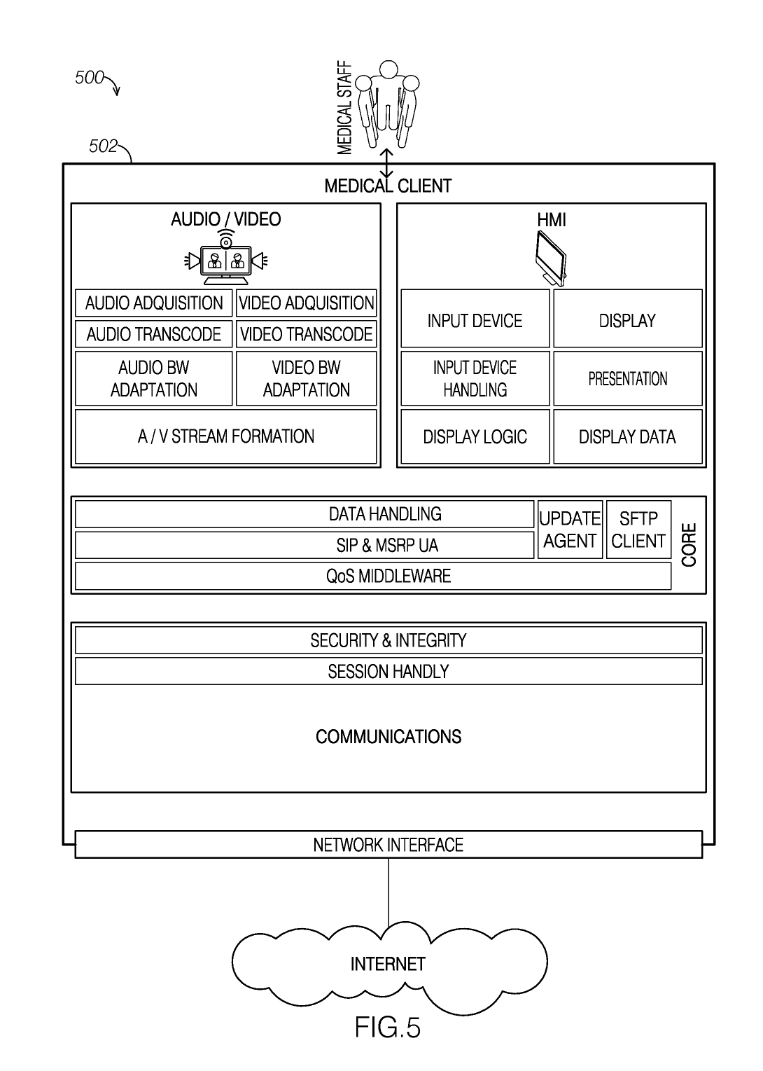

FIG. 3 shows a block diagram of the patient monitor architecture 300 according to one embodiment. FIG. 4 shows a block diagram of the cloud infrastructure architecture 400 according to one embodiment. FIG. 5 shows a block diagram of the cloud medical client architecture 500 according to one embodiment.

According to a particular embodiment, the portable medical apparatus comprises: (a) a patient monitor 302 comprising a plurality of wireless biomedical sensors 310 including an electrocardiogram sensor 312, a non-invasive blood pressure sensor 313, and a pulse oximetry sensor 314; and (b) a communications module 360 configured to wirelessly transmit jointly compressed signals. The communications module is configured to transmit signals as a block of coherent data. Additionally, the communications module includes fast-joint coding and decoding of said signals, transmission error correction, it is configured to enable information exchange between different layers to optimize network throughput, and adapts the Quality of Service (QoS) guarantees for each type of traffic offered. Each layer in the communications module obtains information features about the channel conditions during transmission and said layer processes are adapted to said conditions during transmission and it employs cross-layer protocol interactions. In a particular embodiment the communications module is not based on the Open Systems Interconnection (OSI) network design, but employs Joint Source Channel Coding (JSCC) and a rate controller configured to take feedback from a source coder, a channel coder, and a channel decoder, and allocate an overall rate between said source coder and the channel coder based on real-time performance demands. In some embodiments the JSCC is modified to use a tandem structure that distributes the channel capacity to the source, and channel coder and the communications module employs hierarchical modulation and a cluster progressive source encoder and decoder. The communications module includes an encryption module. Particular embodiments of the encryption module employ a Chaos Video Encryption Scheme (CVES). The following sections provide additional detail regarding these features and embodiments.

B. Multidata Collection and Transmission

There are many challenges associated with the use of interactive collaborative environments. As an example, the MPEG-2/MPEG-4 functionalities need to be redesigned in the context of synchronized and jointly compressed signals. Users may be reviewing a particular signal, asking to see the corresponding signals (images or video) from other modalities. Consequently, the system incorporates fast joint-decoding methods for interactive preview. According to one embodiment, for real-time collaborative work, the heterogeneity of the networks, computing systems and image displays are scalable, network-aware systems. The system implements synchronization of biomedical signals and the supporting data, as well as transmission error correction.

The main challenge in communications is trying to convey as much information as possible over a given channel with as few errors as possible. Shannon's theorem states that a source with entropy H can be reliably transmitted over a channel with a capacity C as long as H≦C. The independence between source and channel coder is the reason why this theorem is also known as the separation theorem. This independence permits simplifying the construction as well as changing any coder (either the source or the channel) while leaving the other unchanged. However, independence between source and channel coder is not always the best approach, especially when streaming video over wireless communication. This traditional approach has some drawbacks: 1) it is necessary to allow infinite complexity and delay in the coders in order to reach optimality (which is problematic for real-time communication), 2) the theorem is not valid for non-ergodic and multi-user channels, and in those cases we no longer have an optimal system, and 3) such systems tend to break down completely when the channel quality falls under a certain threshold, and the channel code is no longer capable of correcting the errors. This phenomenon is often referred to as the “threshold effect.” Consequently, according to one embodiment, the system tries to reduce the threshold effect, since wireless channels have fluctuating channel qualities and high bit error rates. In a particular embodiment, this is accomplished by employing joint source-channel coding.

According to one embodiment the system incorporates a robust, secure and effective method to transmit video, images, and bio-signals. In particular, it incorporates:
Synchronization: When medical data is transmitted (video, voice, bio-signals, etc), the data is synchronized in order to be received as a block of both coherent and related data.
Fast-joint decoding: Related to real-time, the process for coding/decoding data has to be close to real-time. Consequently, in terms of source and channel coding, fast-joint decoding is implemented.
Transmission error corrections: In order to guarantee the robust and secure features, a method to achieve error corrections during a transmission is implemented to provide for error resilience and error concealment.

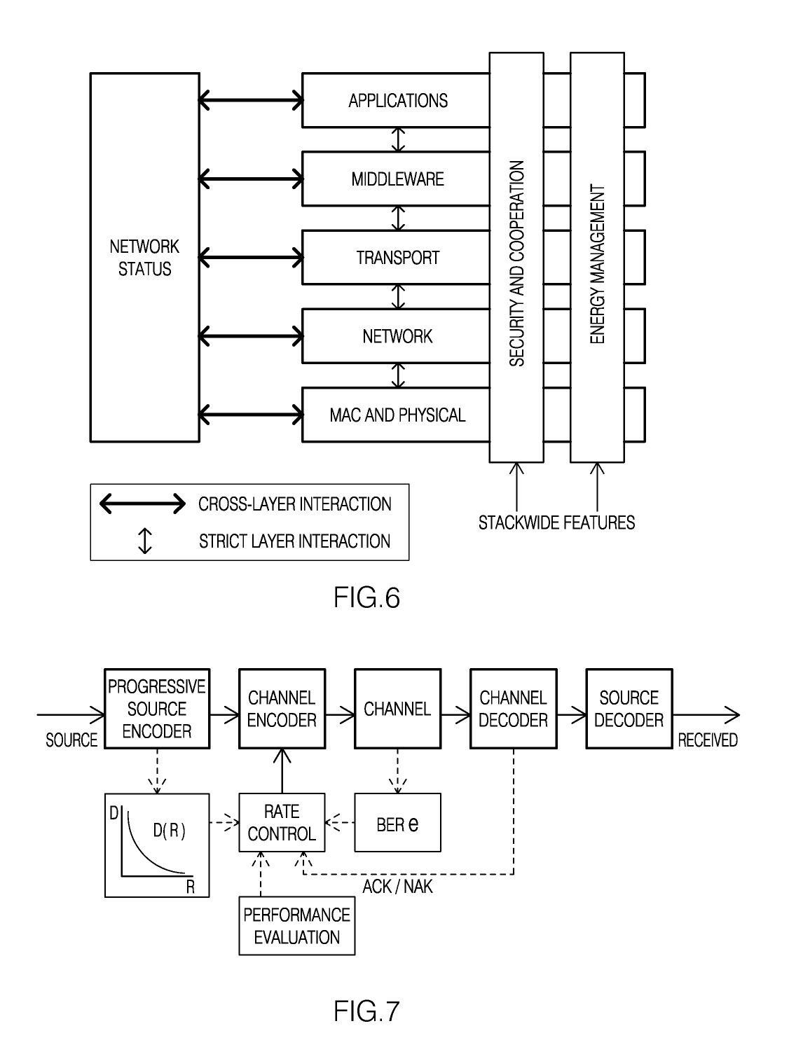

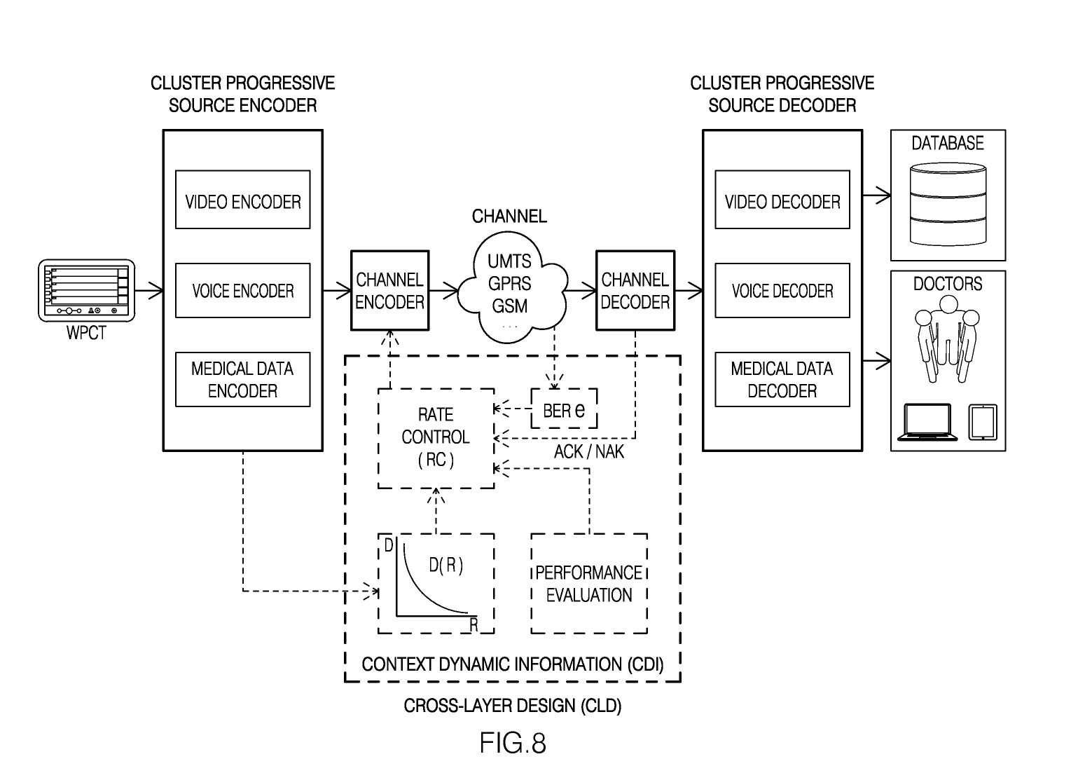

According to one embodiment, as shown in FIG. 6 and FIG. 8, the system employs Cross-Layer Design for transmitting the data (i.e. ECG signals, voice and video) over different and arbitrary noisy channels like wireless, 3G, GPRS and so on. The Cross-Layer Design (CLD) is important for networks based on wireless technologies, since the state of the physical medium can significantly vary over time. Additionally information exchange between different layers can optimize the network throughput. Additionally, the system is designed to adapt the Quality of Service (QoS) guarantees for the different types of offered traffic. The traditional network stack design—the OSI stack—was developed for a very general purpose and, in fact, is really a reference model. Transfer Control Protocol/Internet Protocol version 4 (TCP/IPv4) is today the most successful implementation of the OSI reference model, however, it also inherits its potential flaws and weaknesses for this particular application. For instance, the stack design is highly rigid and strict, and each layer focuses only about the layer directly above it or the one directly below it. This results in a non existent collaboration between the different layers. Additionally, calculated “features” in any IP based network such as packet loss, retransmission, packet congestion, routing problems, reassembly trouble and timeout are highly common, and not well suited for real-time application deployment. Real-time applications in a cellular network, relying on packet switched infrastructure, proved to be problematic. Consequently, according to one embodiment, the system uses CLD since it offers each layer knowing features about current conditions of the channel, and thus each layer may adapt its process to the current channel conditions improving the QoS for real-time communication in each case. The system uses cross-layer protocol interactions to increase network efficiency and better QoS support.

Traditionally, source and channel coding have been addressed as independent problems. Source coding aims to remove redundancy using an efficient representation of the source signal. Channel coding involves adding redundancy to achieve error free transmission in noisy environments. Shannon's separation theorem states that source coding and channel coding can be done separately and sequentially without loss of optimality. However, for Shannon's separation theorem to be truly optimal, infinite block length codes have to be used, which induces infinite complexity and delay. Such requirement makes Separate Source Channel Coding (SSCC) approach problematic for this application. Furthermore, SSCC is designed for the worst case scenario. This means available resources are wasted if and when the channel is good. Similarly, when the channel state is worse than what the channel code is designed for, the system performance collapses and the BER can increase exponentially. Additionally, SSCC is not optimal for multi-user and non-ergodic channel environments. Consequently, according to one embodiment, the system employs a Joint Source Channel Coding (JSCC) approach to share information between the source coder and the channel coder, and utilizes the soft information from the physical layer, instead of treating the source and the channel code as independent blocks.

According to one embodiment, as shown in FIG. 7, the Rate Controller (RC) takes feedback from both the source coder, the channel and channel decoder, and optimally allocates the overall rate between the source and channel coder under preset performance demands (e.g., from a QoS demand from the user or network provider). In particular embodiments, and without limitation, JSCC techniques include: source optimized channel coding, channel optimized source coding, iterative algorithms, channel codes for compression and error protection, and Shannon mappings. JSCC allows the coder(s) to better exploit the changes in the channel conditions or variations of the source contents. As a result, the system has more adaptive and robust methods which give better performance when operating under delay constraints or time varying channels.

In a particular embodiment of the system, the system employs a modified JSCC method that uses the tandem structure, but instead of fixing the rates to the coders, it distributes the channel capacity to the source and channel coder. In one embodiment the scheme grants more bits to the channel coder (and fewer to the source coder) when the channel is bad in order to avoid breakdown in the source decoder, and allocate more bits to the source coder when the channel is good in order to improve the quality. In this implementation, the system is not technically based on JSSC, since the coders are not matched in any sense, but its parameters are modified as the channel quality is changing.

According to a particular embodiment, the system uses “hierarchical modulation” to protect important parts of the data better by organizing the modulation space properly. The wavelet transform used in the JPEG2000 standard is a so-called multiresolution coder where the image is split into different bins in which information content ranges from coarse to fine. By pairing such a multiresolution source coder with a multiresolution modulation scheme, the system enables the receiver to decode the received signal to a resolution/quality depending on the channel signal-to-noise ratio (CSNR). Consequently, the better the CSNR, the better the decoded signal.

FIG. 8 shows the multi-data collection and transmission architecture. This particular embodiment, and without limitation, is based on a progressive JSCC architecture but adding a “Cluster Progressive Source Encoder/Decoder.” Both channel conditions and source coding rate are shared along layers. In particular embodiments, a progressive bitstream is decoded as it arrives, providing a continually improving approximation to the decoded signal. According to one embodiment, transmitting video and streaming data (in this case medical data, i.e., ECGs and so on) is based on JSCC combining source-channel coding for “rate allocation.”

According to one embodiment, as shown in FIG. 8, the “Context Dynamic Information” (CDI) section is formed by a Rate Controller (RC) that takes feedback from both the source coder, channel and channel decoder, and optimally allocates the overall rate between the source and channel coder under preset performance demands, for example, from a QoS demand from the user or network provider. Related to the encoder, the operational rate-distortion curve (function D (R)) is always available for each transmitted source. Thus, given the source rate-distortion curve and the statistical properties of the channel, the aim is to determine the channel coding rate that will give the best end-to-end quality, according to a distortion criterion. Additionally, embodiments of the system also synchronize the sources by transmitting sources as a block of related data. In one embodiment, this is accomplished by using a Cluster Progressive Source Coder. This extended progressive coder permits the system to encode different types of data that are produced at the same time and, therefore, they must be transmitted jointly as a self-block of data, and decoded as a same block of related data at the receiver as well. This novel approach ensures synchronization of different data sources which add to the capabilities JSCC provides itself, achieving an advanced emergency communication model.

According to one embodiment, the system is optimized for transmission of emergency medical data over time-varying and noisy channels in real-time by a) implementing a “Synchronization” method based on using a Cluster Progressive Source Coder to synchronize “blocks of related data,” b) implementing a “Fast-joint decoding” method operating under a delay-constraint (i.e., real-time) on a time-varying channel by jointly optimizing both the source and the channel coders (applying a joint source-channel coding (JSCC) methodology and cross-layer design, and c) a method for “Transmission error corrections” where the feedback information and protocols ensure error corrections.

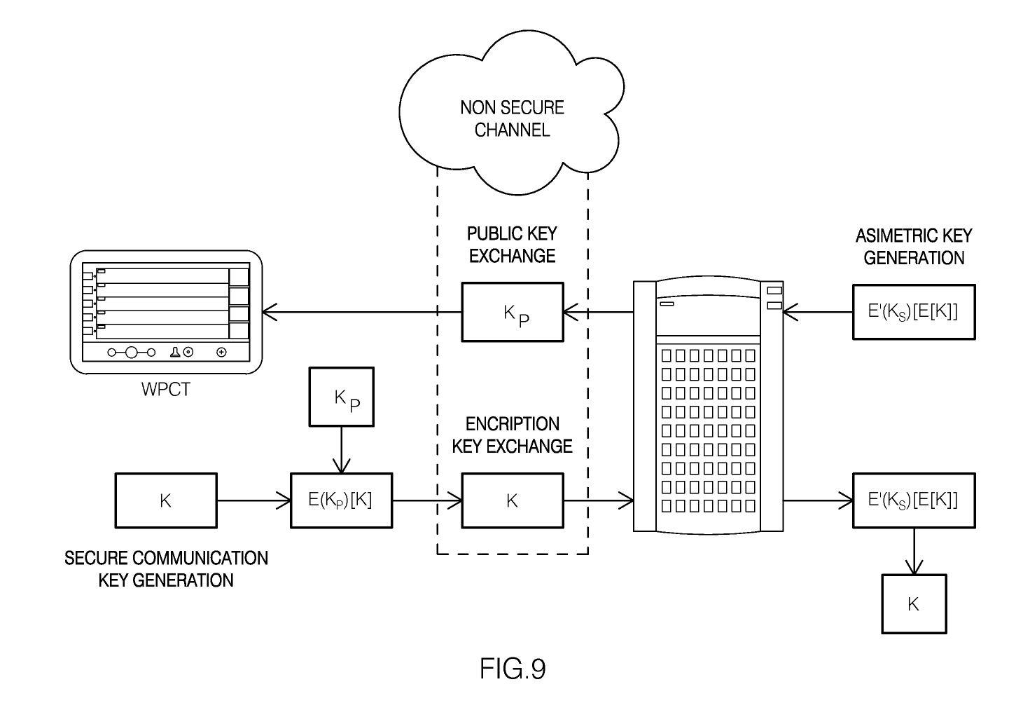

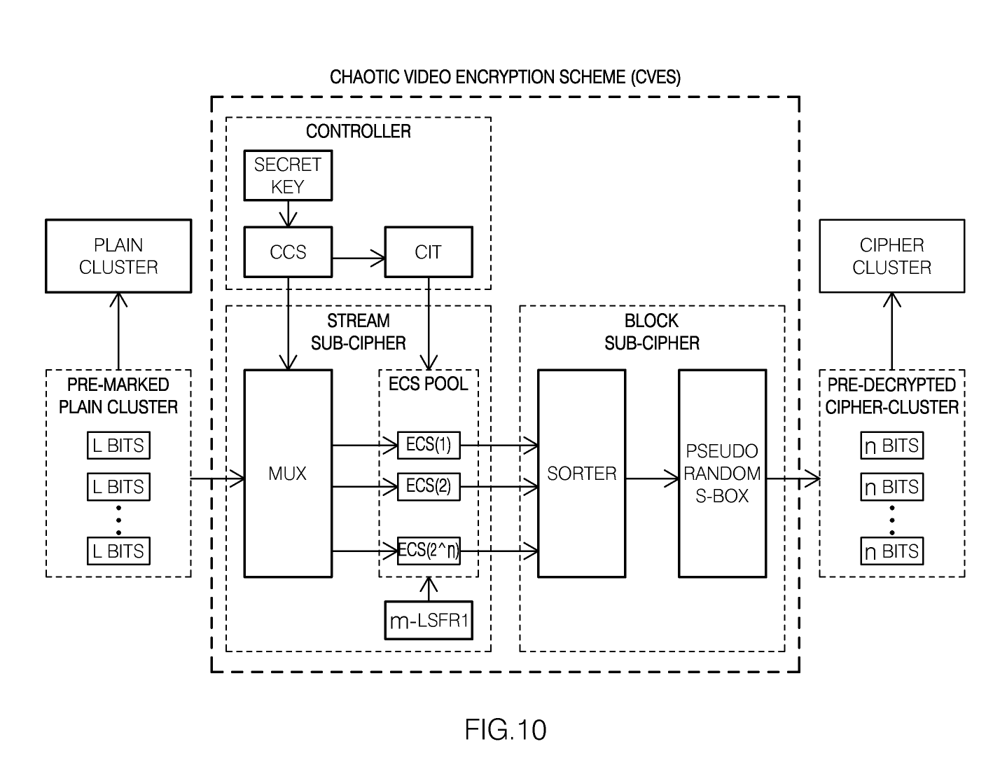

According to one embodiment, the encryption scheme provides a secret key to control the encryption of the plain data to cipher. The patient monitoring device generates a different secret key previous to any exchange of sensible data. Any public-key algorithm is used to secure the communication key exchange. According to a particular embodiment, and without limitation, the system implements a “Chaos Video Encryption Scheme (CVES)” including product cipher of a chaotic stream sub-cipher and a chaotic block sub-cipher. FIG. 9 and FIG. 10 show the architecture of encryption scheme according to a particular embodiment. In an embodiment, instead of using a single stream cipher to generate pseudo-random numbers to mask the plain text, which weakens the capability to resist potential attacks, it uses multiple chaotic systems as chaotic stream ciphers since they are faster than chaotic block ciphers. The entirely encryption scheme is used to reduce the large number of iterations in the block cipher to make the ciphertext independent of the plaintext, which leads to a faster encryption speed. CVES supports sequential retrieval: to decrypt a cipher cluster it must decrypt the previous cipher clusters. In one embodiment, random retrieval functionality is implemented by generating a rank sequence to control the chaotic iterations of the ECS-es and adding a reset mechanism in the ECS-es. According to one embodiment, the the encryption scheme comprises the following modules:
Controller: obtains the initial conditions and parameters for the stream sub-cipher:
   CCS: digital Control Chaotic System used to initialize the stream sub-cipher and control the chaotic iterations of the 2n ECS-es. The control parameter and initial condition of this system are extracted from the secret key generated in each attention.
   CIT: Control Information Table used to store the required information in CVES.
Stream Sub-Cipher: encrypts the plain cluster into the pre-masked plain-cluster:
   ECS Pool: 2n digital Encryption Chaotic Systems
   M-LSFR: used as the perturbing PRNG for all the ECS-es.
   2n×1 MUX: controlled by CCS, selects an ECS generating a key to XOR with the plain cluster in blocks of L bits.
Block Sub-Cipher: substitutes the pre-marked plain cluster into the cipher cluster:
   Sorter: mixes the pre-marked plain cluster
   Pseudo-Random S-Box: controlled by the current states of the ECS-es, obscures the relationship between the key and the ciphertext.

According to one embodiment, the encryption/decryption procedure is as follows: the initial condition and control parameter for CCS are extracted from the secret key which is iterated 2n times to obtain the initial conditions for each ECS, and again 2n times to obtain their control parameters. Control parameters are ranked to generate a sequence to control the chaotic iterations of the ECS-es. The plain cluster is divided into blocks of L bits of data. For each block an ECS would be selected according to the order specified by the rank sequence, and iterated once to encrypt it. The encryption procedure continues until the last block in the plain-cluster. Then the pre-masked plain-cluster is sent to the block sub-cipher which is a substitution cipher with time-variant S-Box controlled by the states of the ECS-es after encrypting a block of data. Decryption is the inverse of the encryption procedure. A cipher-cluster is decrypted by the block sub-cipher, being the S-Box the inverse of the one used for encryption.

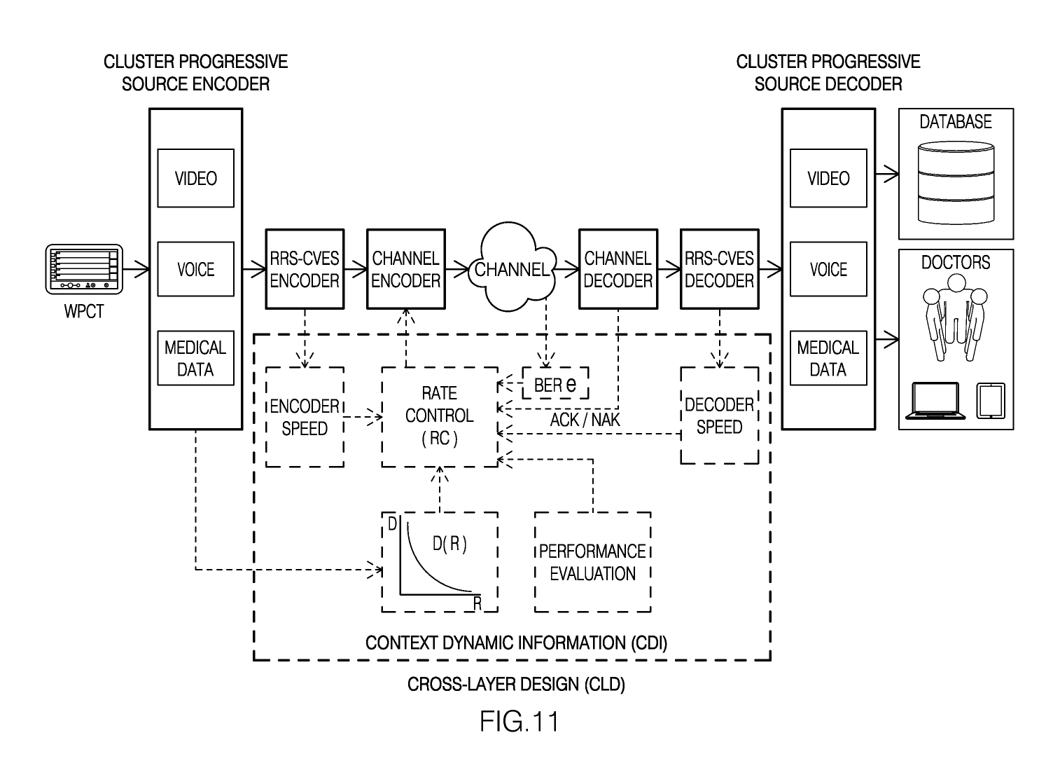

FIG. 11 shows the integration of the encryption scheme into the transmission system architecture according to one embodiment. The encryption scheme runs after a block of multimedia related data is synchronized by the “cluster progressive source encoder,” providing the “Context Dynamic Information” (CDI) section feedback from the encryption speed. In the receiver side, data is decrypted using the inverse scheme used for encryption and information of the decryption speed will also be provided to the CDI.

C. Connectivity

According to one embodiment, the system provides connectivity RTD in BEAT and 4G compatibility. The system provides location-awareness and vertical mobility management. In one particular embodiment, and without limitation, the system utilizes the asymmetric data rates in overlapping heterogeneous wireless networks to improve performance. The handoff algorithm takes into account both moving-in and moving-out scenarios.

In one embodiment, the system includes a vertical mobility and handoff method in a packet-switched all-IP empirical context. This is accomplished by means of a middleware method that 1) looks up in the protocol stack at the application layer requirements from applications such as Voice over IP (VoIP) or mobile file sharing, and 2) looks down in the protocol stack and sees the underlying wireless radio network resources. The overall system incorporates handoff algorithms, mobility management, and mobile middleware.

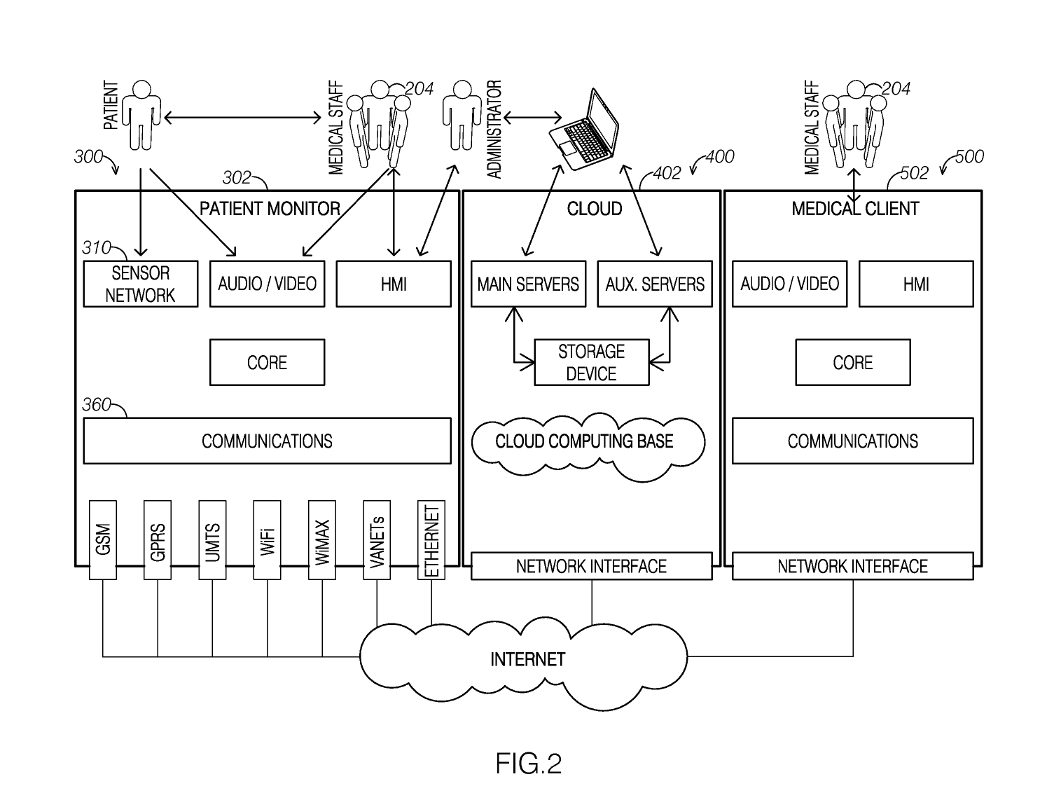

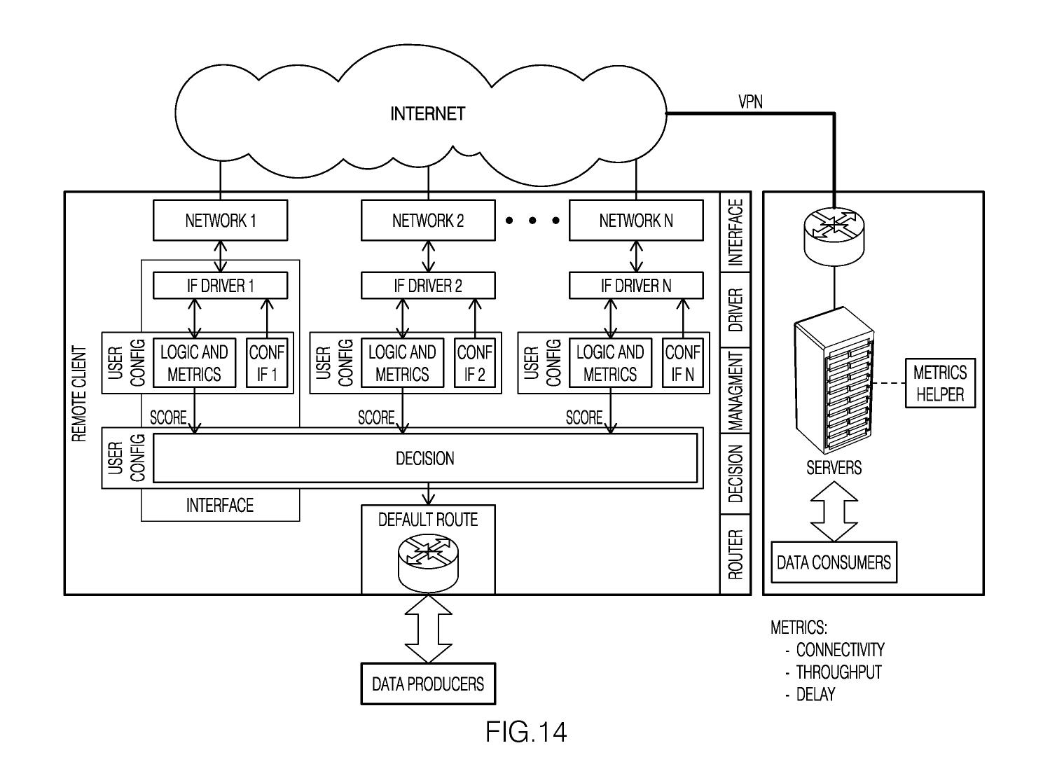

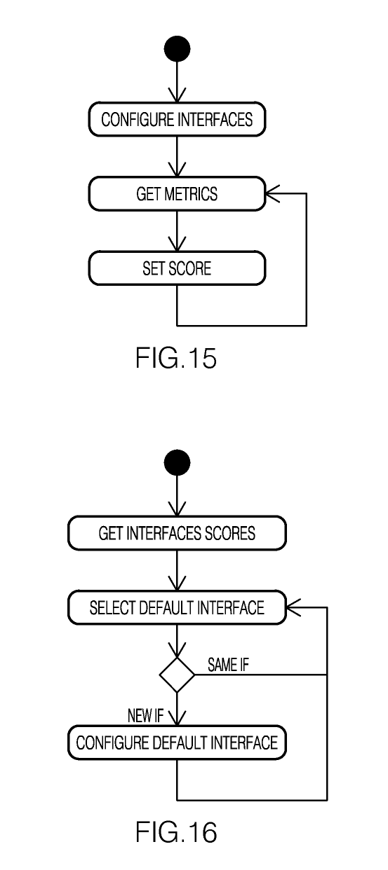

According to one embodiment, as shown in FIG. 1-5 and FIG. 14, the system includes seamless roaming and hand off. FIG. 13 shows the state diagram to manage the interface.

According to one embodiment, and as described above, the system implements a reliable and secure method to transmit the medical data. Most conventional ciphers such as RSA or DES are a good choice when non real-time requirements apply, but due to their low encryption speed cannot be directly used in real-time systems, especially when they are realized by software. Furthermore, their difficulty to incorporate them into the whole system when different video/image compression algorithms can be used creates the necessity of developing specific encryption schemes. Consequently, according to one embodiment, the system implements a method for securing multimedia data that 1) supports any type of data: video, images, biosignals, etc, 2) provides high security with a low encryption processing time, and 3) is independent of any compression method. In a particular embodiment, and without limitation, the system is based on a “Random-Retrieval-Supported Chaotic Video Encryption Scheme (RRS-CVES).” This cipher is especially designed to fulfill needs of real-time video encryption and provides fast encryption speed and high level of security simultaneously.

D. Semantic Middleware Architecture

Existing network and state of the art frameworks do not fulfill the requirements for ubiquitous environments, (i.e., although some specific techniques for monitoring and event correlation, service discovery, quality of service and policy-based management already exists, there is not a general solution that tackles all these features). Current frameworks are mostly aimed at large-scale corporate environments, telecommunications networks and Internet service providers. Their architecture is based on functional decomposition where the various functions are integrated in centralized network operations centers managed by human administrators.

According to one embodiment, and without limitation, the system employs an autonomous middleware that incorporates information from external applications and creates peer-to-peer collaboration relationships. The middleware in the system is common for all applications, which facilitates the semantic interoperability between different biomedical devices, security, mobility, context awareness, and the quality of service. Furthermore, new configurations can be established through the change of the middleware, instead of changing every single application. Consequently, such changes are transparent for the end-user.

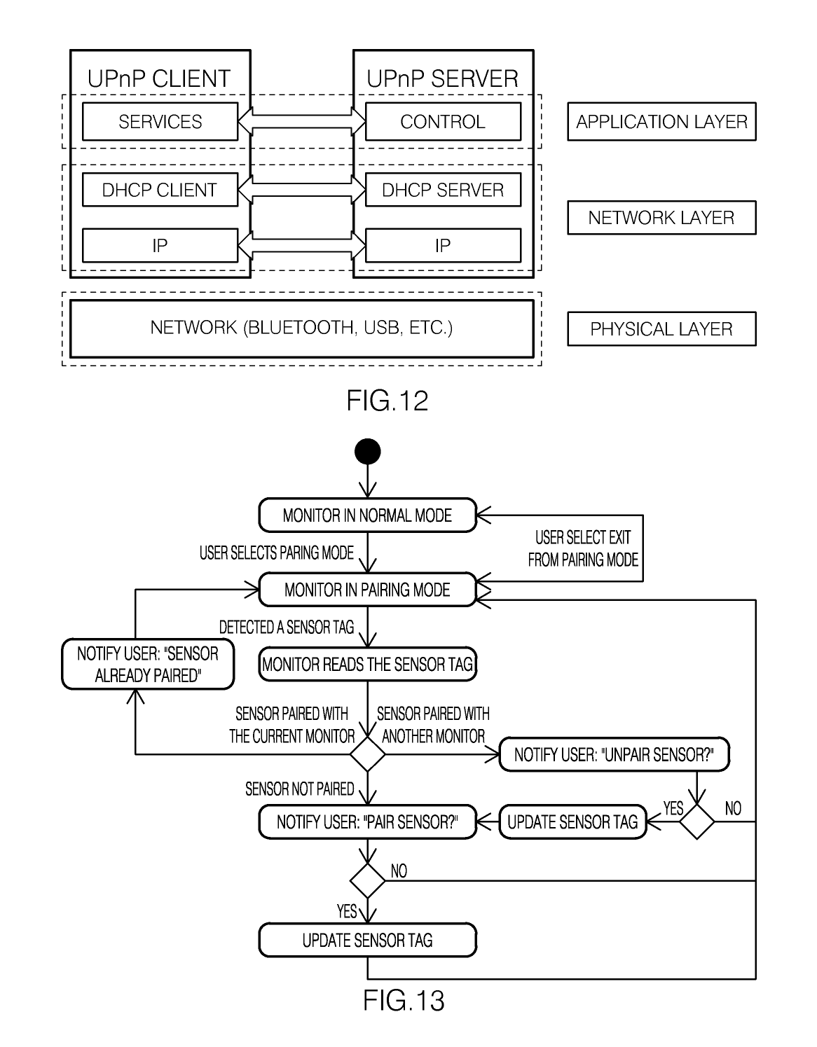

According to one embodiment, as shown in FIG. 12, the semantic middleware architecture is based an UPnP architecture. FIG. 13 shows the state machine with the process of device pair-management according to a particular embodiment.

### Part B—Monitorin System for Stretchers and Hospital Beds

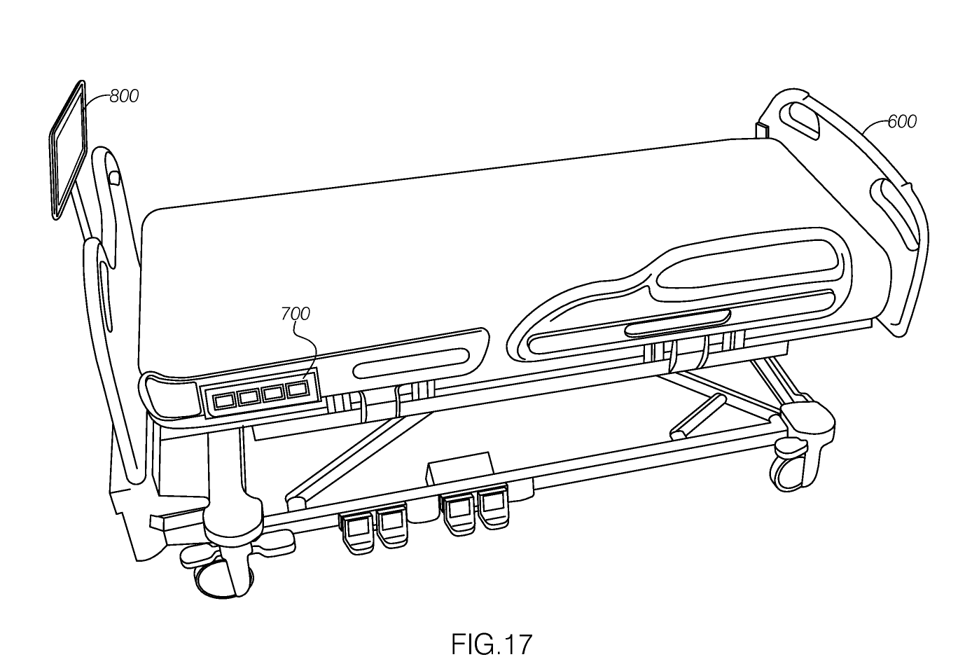

According to one embodiment, the portable medical apparatus comprises: (a) a hospital bed or medical stretcher 600; (b) a plurality of wireless biomedical sensors 700 attached to the hospital bed or medical stretcher; and (c) a communications module configured for wirelessly transmitting jointly compressed signals. In a specific embodiment, the communications module is configured for transmitting said signals as a block of coherent data. In certain embodiments, the communications module is further configured for fast-joint coding and decoding of said signals and transmission error correction. FIG. 17 shows an illustrative embodiment of the wireless patient monitoring system embedded as part of a hospital bed or medical stretcher.

In certain particular embodiments, the communications module is further configured for enabling information exchange between different layers to optimize network throughput and adapting the Quality of Service (QoS) guarantees for each type of traffic offered. Additionally, the communication module can be further configured for obtaining information features about channel conditions during transmission and adapting layer processes to said channel conditions during transmission. It can also employ cross-layer protocol interactions. In one particular design, the communications module 1) is not based on the Open Systems Interconnection (OSI) network design; 2) employs Joint Source Channel Coding (JSCC); and 3) employs a rate controller configured for taking feedback from a source coder, a channel coder, and a channel decoder to allocate an overall rate between said source coder and said channel coder based on real-time performance demands. In a specific embodiment, the JSCC is modified to use a tandem structure configured for distributing the channel capacity to the source and channel coder; the communications module employs hierarchical modulation and a cluster progressive source encoder and decoder; and the communications module includes an encryption module implementing a Chaos Video Encryption Scheme (CVES) including a controller, a stream sub-cipher, and a block sub-cypher.

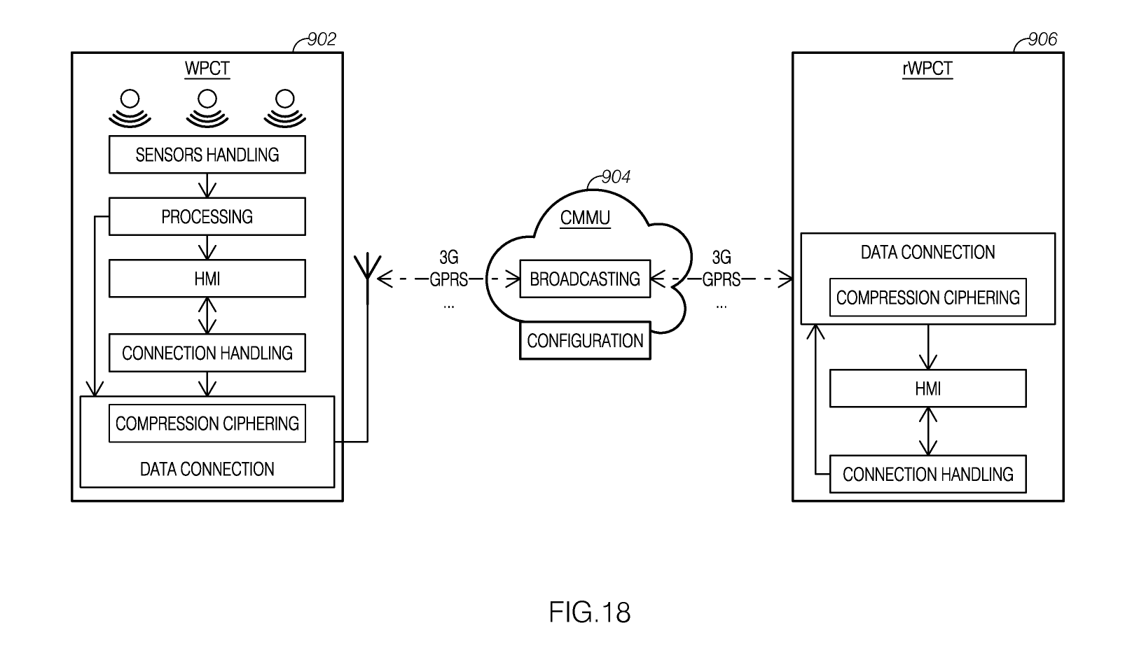

In a particular embodiment, the portable medical apparatus comprises a wireless patient care terminal (WPCT) 902, a central monitoring medical unit (CMMU) 904, and a remote wireless patient care terminal (rWPCT) 906. FIG. 18 shows an illustrative embodiment of the wireless patient monitoring system including the Wireless Patient Care Terminal (WPCT) module 902, the Central Monitoring Medical Unit (CMMU) 904, and the remote Wireless Patient Care Terminal (rWPCT) 906. The WPCT 902 is configured for acquiring, processing, presenting, and transmitting biomedical signals and comprises 1) an early monitoring apparatus including a rack of wireless biomedical sensors and a module for signal processing; 2) a multitouch hardware 800 with an embedded system application, and 3) a connectivity platform configured for data connection, data synchronization, and compression ciphering. The CMMU 904 is configured for data storage and broadcasting and the rWPCT 906 comprises a remote biomedical data viewer for client devices. Client devices include desktops, mobile devices, multitouch tablets, mobile phones, and combinations thereof.

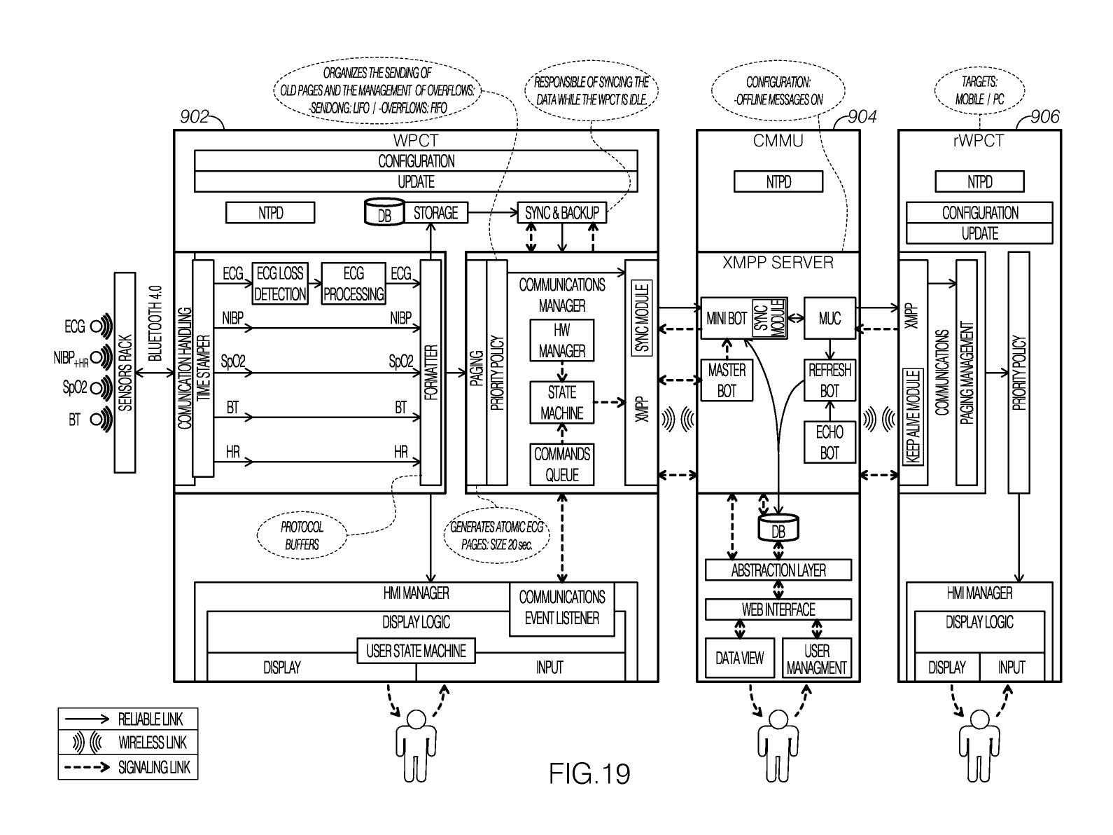

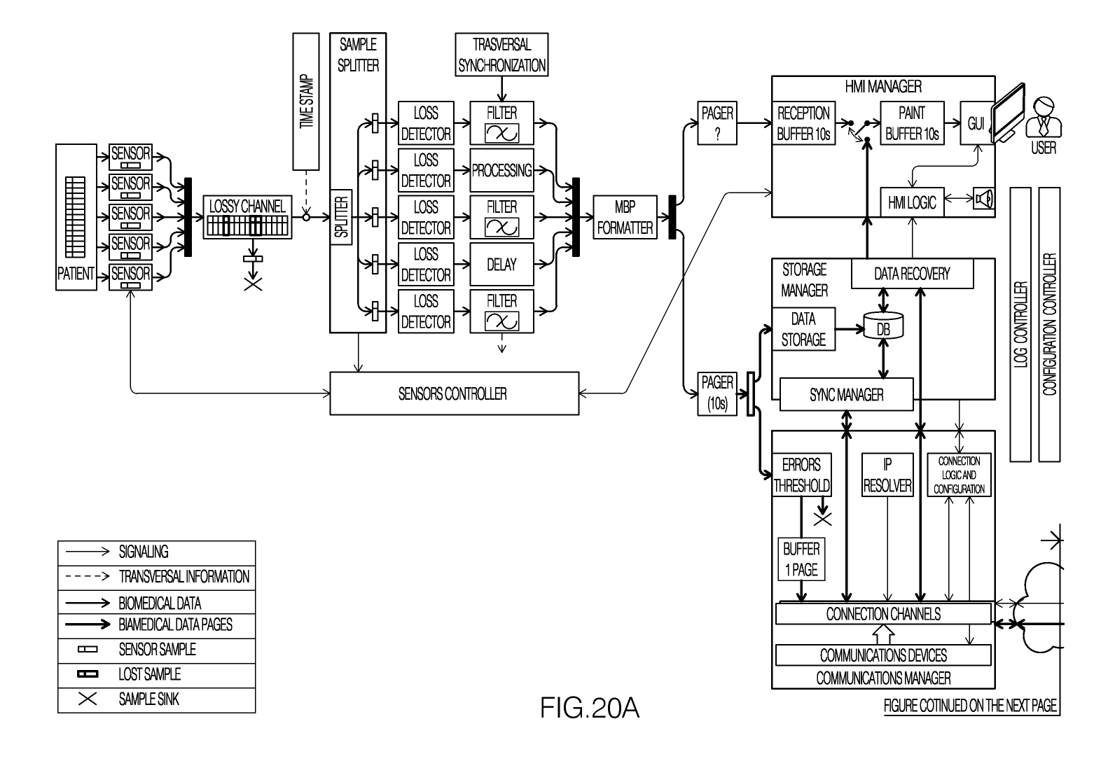

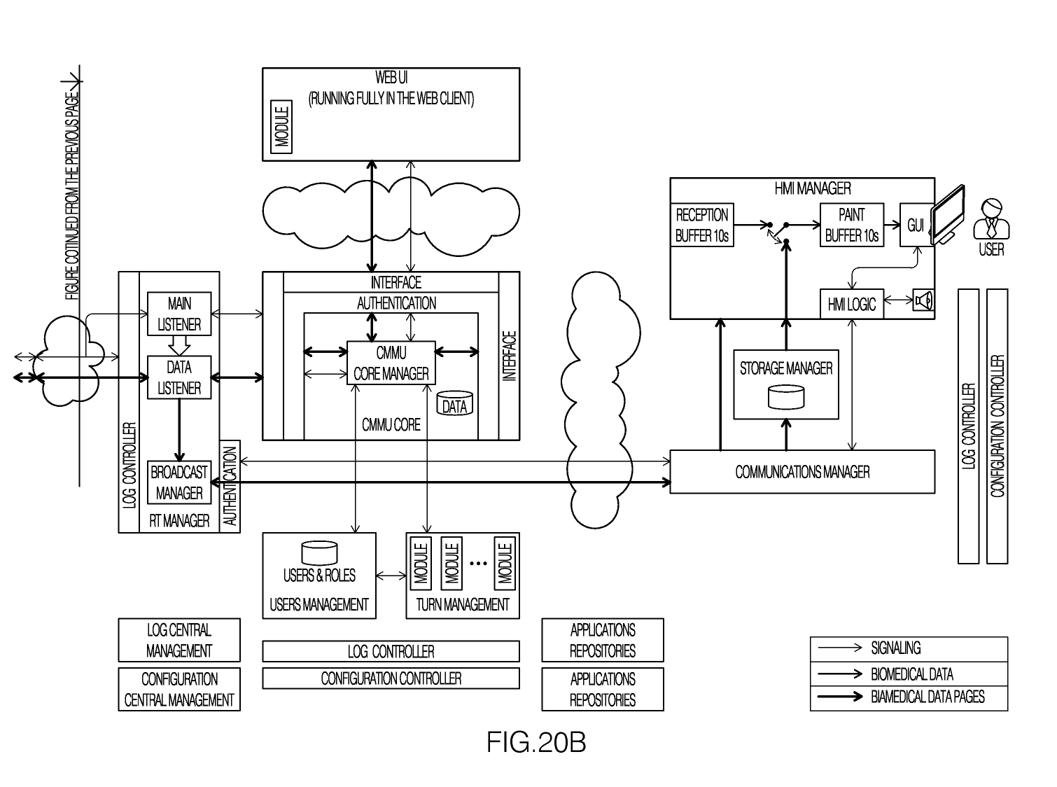

According to one embodiment, the rack of sensors includes a combination of 12 lead ECG, NIBP, SpO2, and respiratory rate BLUETOOTH 4.0 sensors. The sensors include capability to both acquire and transmit the biomedical signals. FIG. 19 shows a block diagram of the overall architecture according to one embodiment and FIG. 20A-20B show a detailed block diagram of the system including signal flows and interconnectivity.

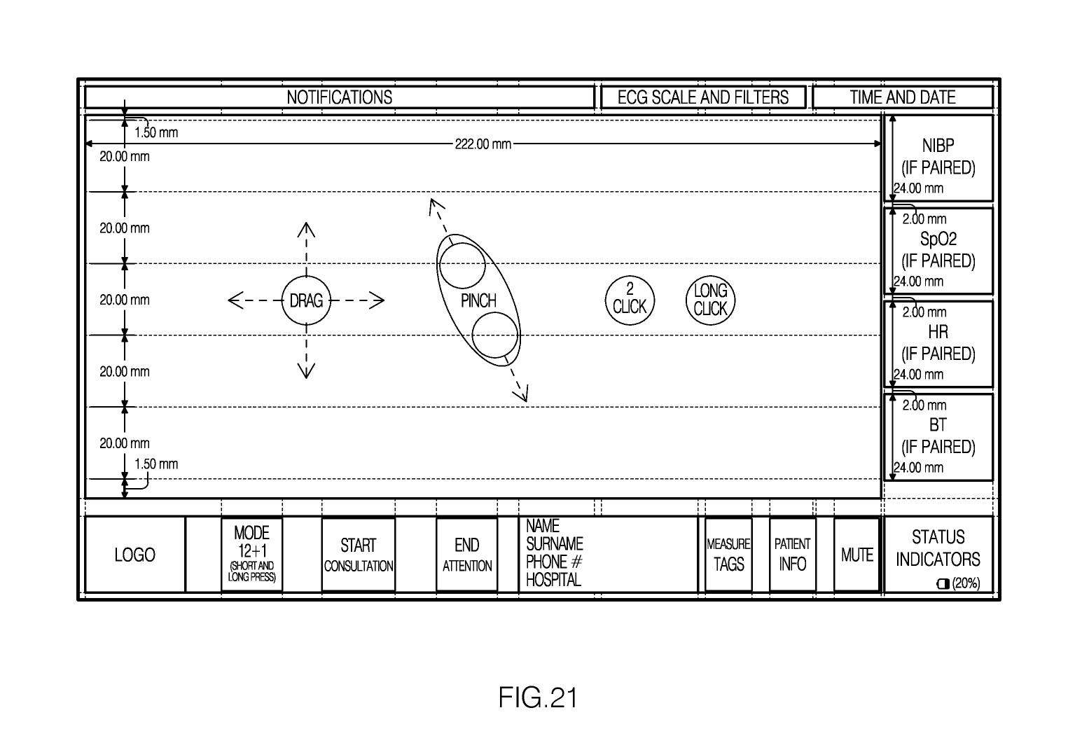

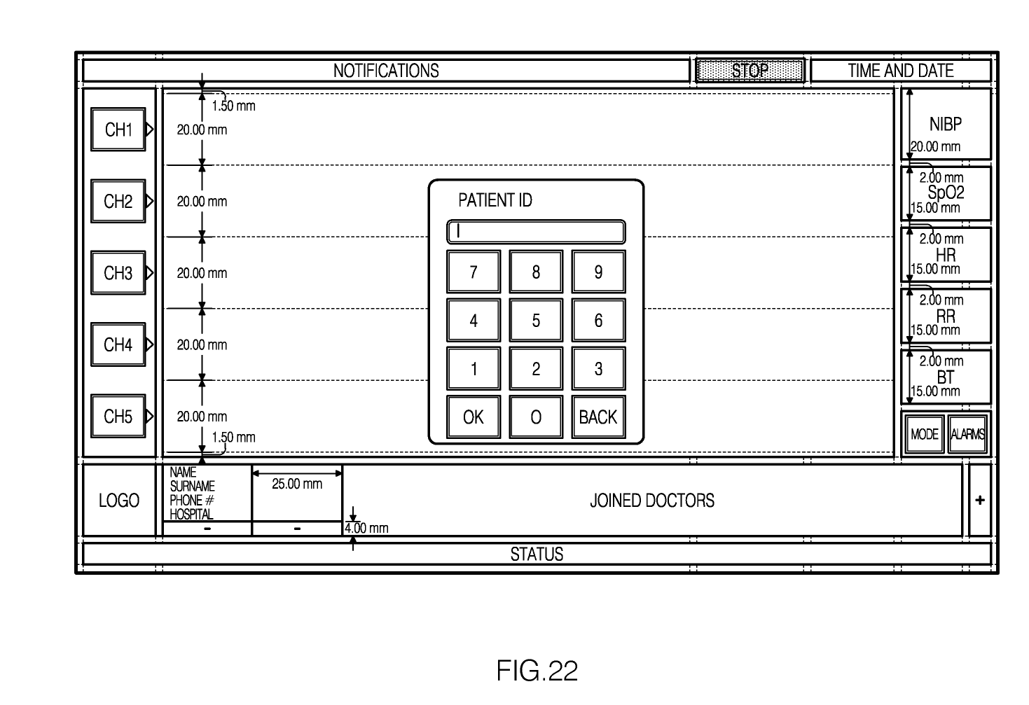

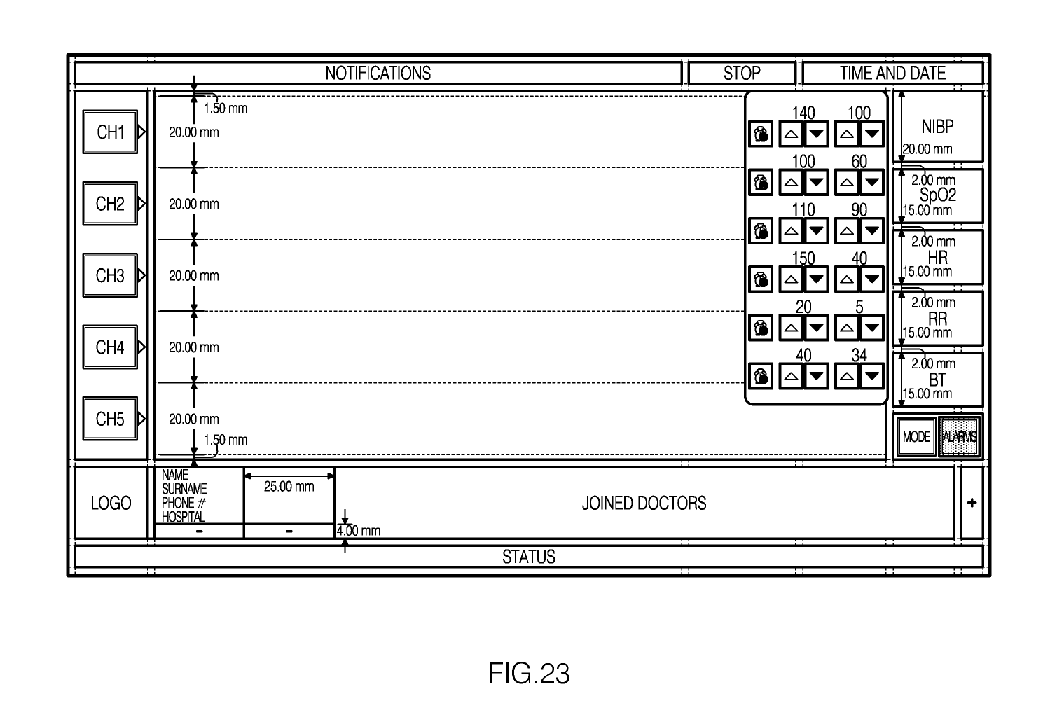

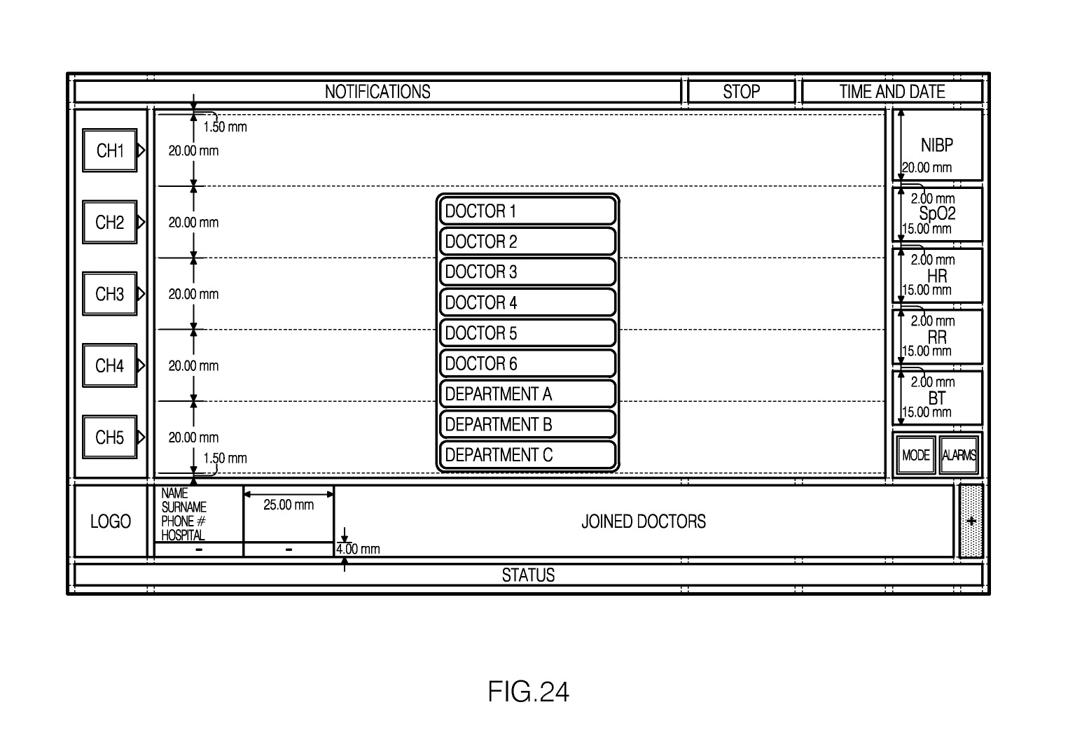

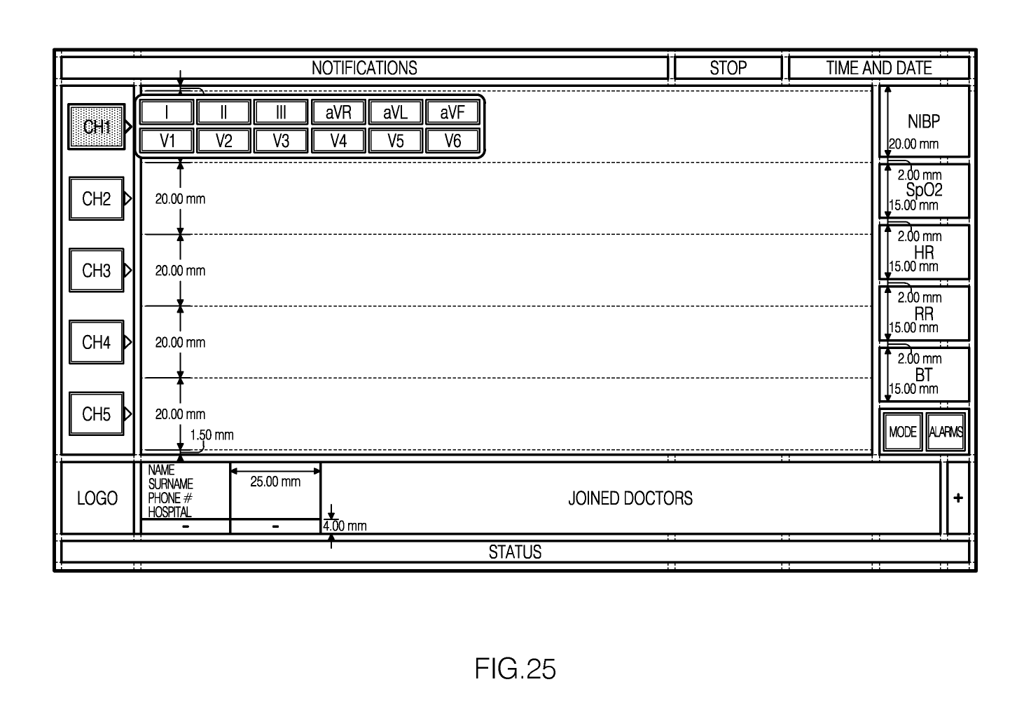

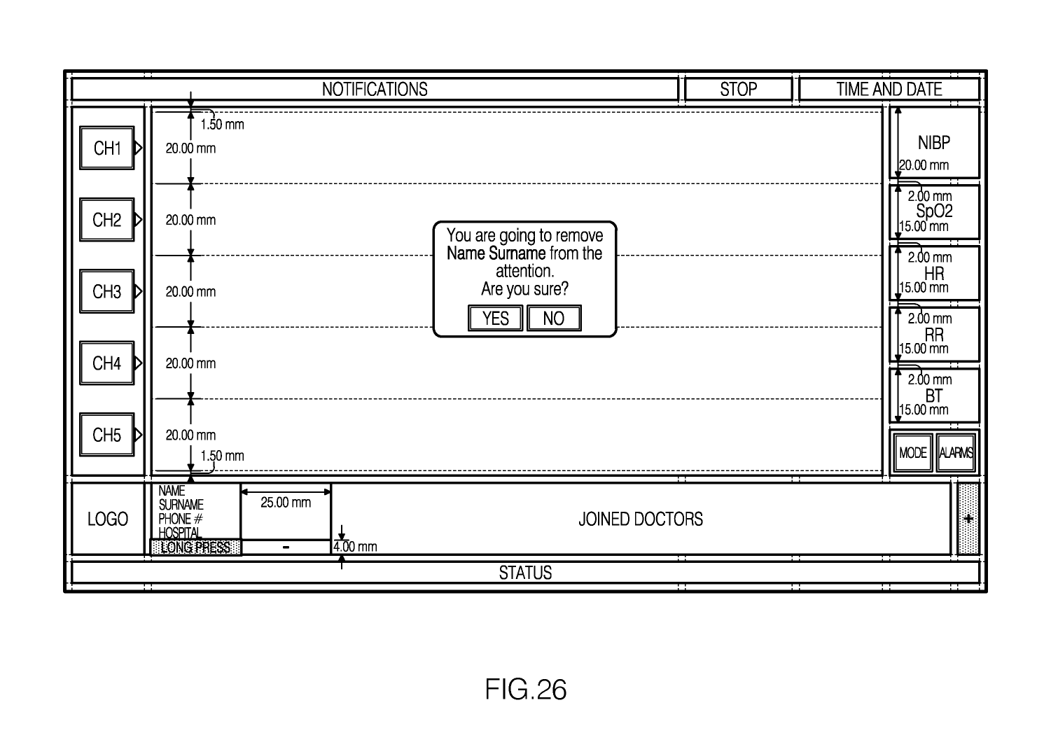

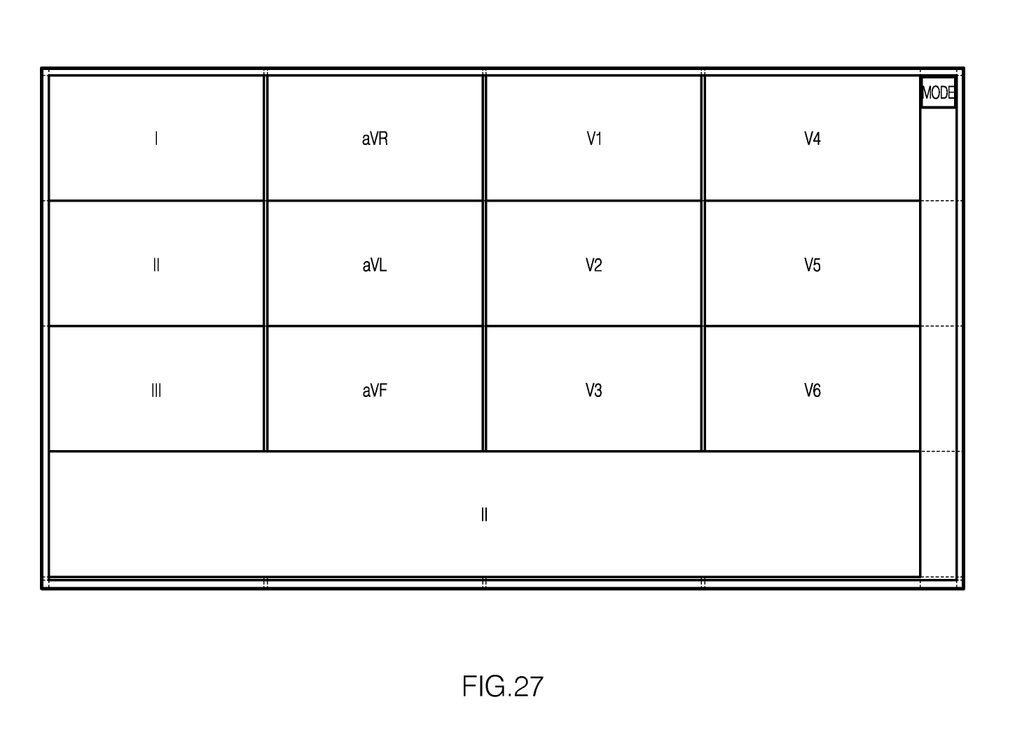

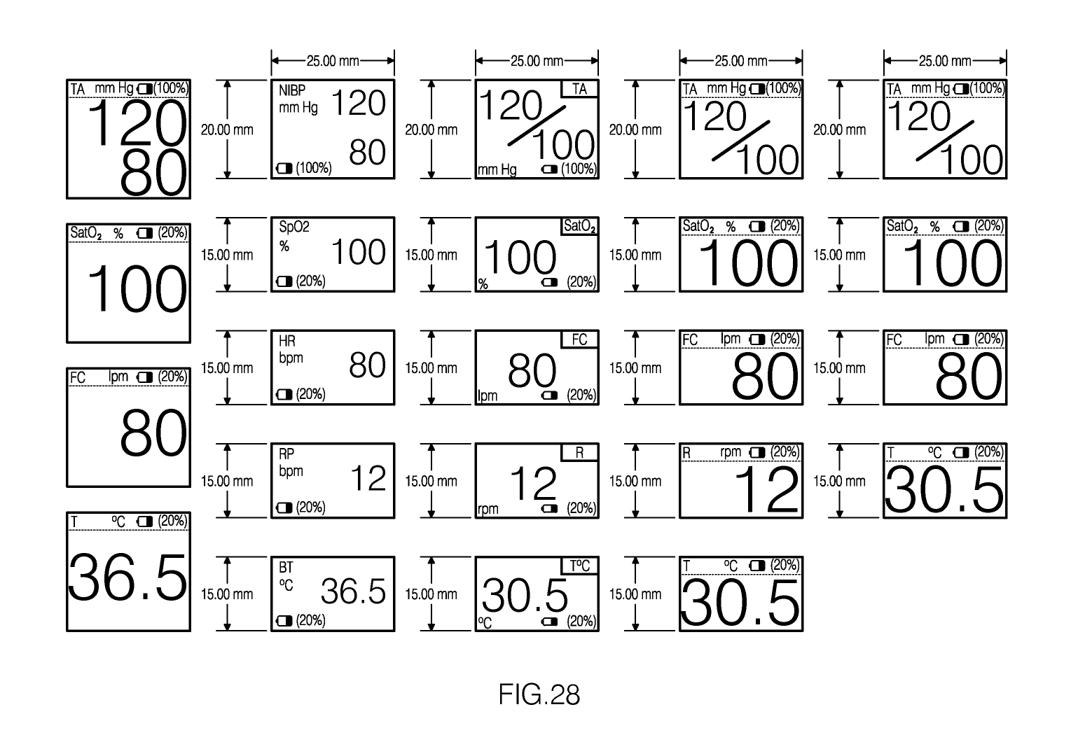

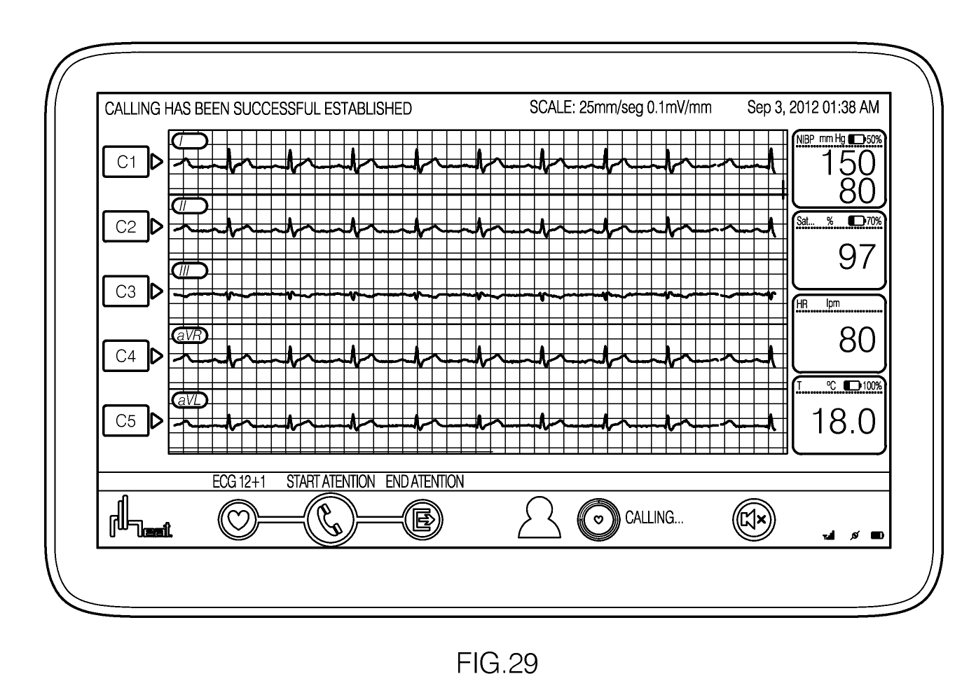

As it can be appreciated in FIG. 17, the system can be used in the same manner as a traditional stretcher or hospital bed. However, the system includes the capability for monitoring the main biomedical parameters in a wireless manner and functionality for extending the presentation of the data to remote medical clients. The data from the wireless sensors and the information coming from the other biomedical equipment is connected to a middleware system (FIG. 20A—Sensors Controller) embedded in the WPCT and then transmitted to the PHR (personal health record), the hospital and to the mobile clients of specialists (if necessary). If the patient needs to be moved to another area of the hospital, monitoring can continue during the move. Each sensor in the bed can be hot-removed and placed in the moving stretcher and transported with the patient. This ensures continuous monitoring since the sensor incorporate long-duration batteries. According to one embodiment, the monitoring unit (WRPCT) is based on specific tablet hardware with a unique touch GUI that facilitates its use for the medical staff (nurses, doctors, caregivers, etc). FIG. 21-28 show illustrative aspects of the graphical user interface (GUI) according to particular embodiments and FIG. 29 shows an illustrative GUI in a multi-touch tablet.

While particular embodiments have been described, it is understood that, after learning the teachings contained in this disclosure, modifications and generalizations will be apparent to those skilled in the art without departing from the spirit of the disclosed embodiments. It is noted that the disclosed embodiments and examples have been provided merely for the purpose of explanation and are in no way to be construed as limiting. While the apparatus has been described with reference to various embodiments, it is understood that the words which have been used herein are words of description and illustration, rather than words of limitation. Further, although the system has been described herein with reference to particular means, materials and embodiments, the actual embodiments are not intended to be limited to the particulars disclosed herein; rather, the system extends to all functionally equivalent structures, methods and uses, such as are within the scope of the appended claims. Those skilled in the art, having the benefit of the teachings of this specification, may effect numerous modifications thereto and changes may be made without departing from the scope and spirit of the disclosed embodiments in its aspects.

The invention claimed is:

1. A portable medical apparatus comprising:
(a) a hospital bed or medical stretcher;
(b) a plurality of wireless biomedical sensors attached to said hospital bed or medical stretcher; and
(c) a communications module configured for wirelessly transmitting jointly compressed signals.

2. The portable medical apparatus of claim 1, wherein said communications module is configured for transmitting said signals as a block of coherent data.

3. The portable medical apparatus of claim 2, wherein said communications module is further configured for fast-joint coding and decoding of said signals.

4. The portable medical apparatus of claim 3, wherein said communications module is further configured for transmission error correction.

5. The portable medical apparatus of claim 4, wherein said communications module is further configured for enabling information exchange between different layers to optimize network throughput and adapting the Quality of Service (QoS) guarantees for each type of traffic offered.

6. The portable medical apparatus of claim 5, wherein said communications module is further configured for obtaining information features about channel conditions during transmission and adapting layer processes to said channel conditions during transmission.

7. The portable medical apparatus of claim 6, wherein said communications module is further configured for employing cross-layer protocol interactions.

8. The portable medical apparatus of claim 7, wherein said communications module 1) is not based on the Open Systems Interconnection (OSI) network design; 2) employs Joint Source Channel Coding (JSCC); and 3) employs a rate controller configured for taking feedback from a source coder, a channel coder, and a channel decoder to allocate an overall rate between said source coder and said channel coder based on real-time performance demands.

9. The portable medical apparatus of claim 8, wherein said JSCC is modified to use a tandem structure configured for distributing the channel capacity to the source and channel coder; said communications module employs hierarchical modulation and a cluster progressive source encoder and decoder; and said communications module includes an encryption module implementing a Chaos Video Encryption Scheme (CVES) including a controller, a stream sub-cipher, and a block sub-cypher.

10. The portable medical apparatus of claim 1, wherein said medical apparatus comprises a wireless patient care terminal (WPCT), a central monitoring medical unit (CMMU), and a remote wireless patient care terminal (rWPCT).

11. The portable medical apparatus of claim 10, wherein said WPCT is configured for acquiring, processing, presenting, and transmitting biomedical signals.

12. The portable medical apparatus of claim 11, wherein said WPCT comprises 1) an early monitoring apparatus including a rack of wireless biomedical sensors and a module for signal processing; 2) a multitouch hardware with an embedded system application, and 3) a connectivity platform configured for data connection, data synchronization, and compression ciphering.

13. The portable medical apparatus of claim 12, wherein said CMMU is configured for data storage and broadcasting.

14. The portable medical apparatus of claim 13, wherein said rWPCT comprises a remote biomedical data viewer for client devices.

15. The portable medical apparatus of claim 14, wherein said client devices include desktops, mobile devices, multitouch tablets, mobile phones, and combinations thereof.
